(19) United States
(12) Patent Application Publication
(10) Pub. No.: US 2004/0241764 A1
(43) Pub. Date: Dec. 2, 2004

(54) Early detection of recipient's immune response against heart transplant

(76) Inventor: Uri Galili, Chicago, IL (US)

Correspondence Address: URI GALILI, Apt. #1404, S. Michigan Avenue, Chicago, IL, 60605, US

(21) Appl. No.: 10/852,325

(22) Filed: May 23, 2004

Related U.S. Application Data
(60) Provisional application No. 60/474,809, filed on Jun. 2, 2003.

(51) Int. Cl. G01N 33/53; G01N 33/567; G01N 33/537; G01N 33/543

(52) U.S. Cl. 435/7.2; 435/7.92

(57) ABSTRACT

A novel noninvasive immunological method for early detection of immune reactivity, primarily antibody reactivity, in heart transplant recipients prior to tissue damage or rejection is disclosed. The method utilizes donor tissue obtained before transplant and processed to obtain an antigen preparation comprising homogenized, fragmented donor cardiac tissue, including the posterior wall of the right atrium and the joining portion of the vena cava. This donor tissue is normally discarded before transplant. The methods include ELISA or light emitting immunoassays for measuring antibody binding. These methods are also applicable to allografts of other organs such as kidney. Such early detection of these immune responses enables a treating physician to modify the immunosuppressive treatment in order to prevent episodes of immune rejection and thus, to prevent damage of the transplanted organ.

### FIELD OF THE INVENTION

The present invention is in the fields of organ transplantation and immunology. The invention describes novel noninvasive and sensitive immunological tests that monitor the anti-allograft immune response in patients transplanted with heart or other allografts. These tests measure the immune response in blood samples against the full range of allo-antigens expressed on the graft.

### DESCRIPTION OF THE BACKGROUND ART

Transplantation of organs from one individual to another member of the same species (allograft) has become one of the routine modes of clinical treatment for patients suffering from impaired organ function. The high success rates of this treatment modality are the direct result of development of effective immunosuppressive drugs that prevent the rejection of grafts expressing antigens that are not present in the transplanted patient. These antigens include primarily major histocompatibility complex (MHC) antigens as well as minor histocompatibility antigens, referred to collectively as alloantigens. The most common type of transplants are of heart and kidney.

Heart Transplantation

Transplantation of a cadaveric heart is performed in patients with end-stage heart disease. The heart is transplanted in an orthotopic position with aortic, pulmonary artery, and pulmonary vein anastomoses. The posterior wall of the right atrium and the joining portion of the vena cava of the donor are removed, and the transplanted heart is joined by a single anastomosis to the retained posterior wall of the recipient's right atrium.

With current immunosuppressive treatments, the 1 year survival rates are excellent. Nevertheless, rejection episodes mediated by immune reaction to the donor's transplantation antigens may occur and can damage the grafted heart. Such rejections may be preceded by fever, tachycardia and heart failure which are the result of these immune reactions. The early stages of such rejection may be diagnosed by routine central venous catheterization and endomyocardial biopsy, or according to clinical symptoms. If rejection is detected, it is treated by corticosteroids and anti-T cell antibodies. However, this protocol, is costly, cannot be used frequently in patients, and may cause complications including pneumothorax, carotid puncture, ventricular perforation and coronary artery fistula. Therefore, the ability to detect a developing rejection process by laboratory immunological tests that measure the specific immune response to the graft will allow for early immunosuppressive intervention that prevents the rejection process from developing to a level that damages the transplanted heart. These tests may reduce and ultimately eliminate the use of endomyocardial biopsy.

Frequent monitoring of immune response to the heart graft may also enable a decrease in immunosuppressive drug doses in individual patients and thus, minimize the detrimental side effects of the immunosuppressive treatment. Immunosuppression results in excessive susceptibility to infections that are responsible for more than 50% of all deaths after heart transplantation. A problematic side effect of cyclosporine (the most commonly used immunosuppressive drug) is exacerbation of coronary atherosclerosis in the graft. Immunosuppression is also associated with malignancy. Both atherosclerosis and malignancy account for 5 to 20% of transplant patient deaths.

Kidney Transplantation

In kidney transplantation, the allograft is placed in a retroperitoneal position against the psoas muscle through an iliac incision. The renal artery is joined by anastomosis to either the internal or external iliac artery and the renal vein is joined to the external iliac vein. The donor ureter is joined by anastomosis to the urinary bladder mucosa. The immunosuppressive protocols used to prevent allograft rejection usually include cyclosporine, azathioprine or cyclophosphamide, corticosteroids and anti-lymphocytic globulin (ALG). All these immunosuppressive drugs are toxic to various tissues. Cyclosporine is toxic to the kidney, azathioprine to the liver, cyclophosphamide to the bone marrow and ALG consists heterologous proteins that can generate an immune response against themselves. Thus, it is desirable to decrease the dose of these drugs while maintaining an immunosuppressive effect adequate to prevent rejection.

The success rate of kidney allografts is associated with the extent of matching of the MHC antigens. The fewer the matched antigens, the stronger is the immune response against the allograft. The symptoms of immune mediated rejection (termed “acute rejection”) include swelling of the allograft and sudden drop in renal function (decreased urine volume and increased blood urea nitrogen and creatinine). About 35% of kidney allograft recipients undergo an episode of acute rejection in the first post transplantation year (Hariharan et al., N Engl J Med 342:605-612, 2000). The kidney may also undergo chronic rejection which can occur months or years post transplantation. This rejection is characterized by narrowing of the blood vessels because of growth of the endothelial cells and fibrotic changes in the blood vessel walls. These chronic changes seem to be associated with an ongoing immune response against the graft and/or toxic effects of immunosuppressive drugs. As in the case of heart allografts, a simple and reliable test for long term monitoring of the immune response against the graft will be beneficial for the adjustment of immunosuppressive drug dose in order to prevent rejection episodes. Alternatively, or additionally, such monitoring may allow gradual decreases in the dose of immunosuppressive agents, thereby reducing the detrimental effect of the drugs, while still preventing the anti-allograft response.

Monitoring Heart Transplants

Monitoring of heart transplant recipients for the development of allograft rejection includes noninvasive methods such as intramyocardial electrocardiogram (Hetzer et al., Ann Thorac Surg 66:1343, 1998) and echocardiography, radioisotope techniques, magnetic resonance imaging and immunological methods (Kemkes et al., J Heart Lung Transplant 11: S221-31, 1992). The immunological methods include the measurement of serum cytokine levels, particularly IL6 and IL8 (Kimball et al., Transplantation 61: 909-15, 1996), monitoring recipient serum for donor HLA antigens and anti-HLA antibodies (Reed et al., Transplantation 61: 566-72, 1996) and measuring reactivity of allo-reactive helper T cells (DeBruyne, Transplantation 56: 722-7, 1993), or cytotoxic T cells in the blood of the recipient (Reader et al. Transplantation 50: 29-33, 1990; Loonen et al., Transplant Int 7:596-598, 1994). The noninvasive methods are associated with the detection of ongoing damage in the heart muscle and thus, may come too late for the preferred goal in improving the care of the transplant recipient: early prevention of the developing rejection episode. Similarly the invasive endomyocardial biopsy detects an ongoing immune rejection process that may have already damaged the heart before immunosuppressive intervention has been initiated.

Monitoring Kidney Transplants

The monitoring methods used in kidney allograft recipients are also directed to detection of damage to the transplanted organ. The invasive method uses needle biopsies. The noninvasive methods include the functional indicators of impaired renal activity, such as (a) decreased urine volume, (b) decreased clearance of creatinine and (c) elevated blood urea nitrogen. Monitoring includes detection of lymphocytes in the urine (Salaman, Immunol Lett 29: 139-12, 1991), secretion of neopterin (a pteridine from stimulated macrophages) and interferon γ (a cytokine released by activated T cells) (Khoss et al. Child Nephrol Urol 9:46-49, 1988; Grebe et al., Curr Drug Metabol 3:189-202, 2002). The sensitivity of detection of inflammatory products in the urine was further improved by measuring the presence of mRNA for perforin and granzyme B (proteins released from T cells that damage target cells), using the reverse transcriptase-polymerase chain reaction (RT-PCR) for amplification and detection of these molecules (Li et al., N Engl J Med 344: 947-954, 2001). Since all these methods depend on the detection of an ongoing destructive immune rejection process in the kidney, the kidney allograft may already be damaged at the time of detection of the rejection episode. As in the case of heart allograft monitoring, the early detection enabled by the invention would result in early prevention of the development of the rejection episode. This will prolong the function of the kidney allograft by early clinical intervention for protection of the allograft from immune assaults.

There is clearly a need in the art for a relatively simple, economical and non-invasive method for detecting graft-specific antibody responses in a transplant recipient prior to the onset of tissue damage which may be irreversible but, in any case, is detrimental. The present inventor has developed such a method, which is described below.

### SUMMARY OF THE INVENTION

The present inventor had discovered and describes herein a simple and highly sensitive noninvasive method that detects the occurrence of an anti-allograft immune response at early stages post-transplant and thus provides the means for clinical intervention that thwarts immune rejection episodes. The method is based in part on the inventor's conception that donor tissue obtained during harvesting and preparation of a heart for transplantation, which is normally discarded, can be saved and used for making an antigen preparation that can be used post-transplant to measure immune responses specific for the grafted tissue.

By the term “early” in relation to the transplant is intended a time after transplantation at which antibodies or reactive T cells may be present and detectable in the blood, but no tissue damage in the transplanted organ, preferably a transplanted heart, is detectable by biopsy and standard monitoring methods. An “early stage” can be as early as 2 weeks post-transplant.

Preferably, more than one serum sample is obtained at various times after transplantation, so that a pattern of development of an antibody response can be observed. Serum samples may also be obtained at later stages, following immunosuppressive therapy, to confirm that the response has been successfully inhibited.

Early prevention of development of rejection episodes in allograft recipients is achieved by noninvasive monitoring of the anti-allograft antibody response, and, optionally, T cell responses, in transplant patients.

The sensitivity of ELISA assays that measure anti-allograft antibody response can be maximized if the immobilized antigen includes the full range of the donor alloantigens. The present inventor therefore developed a method for analyzing such antibodies in recipients of heart allografts using as solid phase (=immobilized) antigen in ELISA, particulate material from cardiac or associated tissue taken from the donor as part of the regular transplant procedure. Most preferred is fragmented (=homogenized) posterior wall of the donor's right atrium and the joining portion of the donor vena cava. Parts of tissue removed from kidney, liver, or other organs may similarly serve as solid phase antigens in evaluating immune responses to transplantation of the corresponding organs. If such tissue, e.g., donor kidney or liver, is not available as a source of antigen for the assay, then donor's lymphocytes (or other cell types) may be immortalized as a cell line by Epstein Barr virus (EBV) transformation or otherwise preserved in long term culture to serve as a source of immobilized antigens for early detection of antibody production against the allograft.

The invention further includes the use of fragmented donor's heart tissue in evaluating heart transplantation patients and of EBV transformed donor's lymphocytes as source of stimulatory allo-antigens for to be used for evaluation of T cell responses (i.e., cellular immune response) against the allograft. The responses of T cells following the stimulation by the donor's allo-antigens are evaluated by measuring stimulation of T cells to proliferate, or produce/secrete cytokines.

By monitoring for early detection of these immune responses, the treating physician may modify the immunosuppressive regimen to prevent episodes of immune rejection and consequent damage of the transplanted organ.

Thus, the present invention provides a method for early detection of an immune response in a transplant recipient against the grafted tissue or organ from a donor, comprising:

(a) testing one or more serum samples from the recipient obtained early after transplantation to measure the level of antibodies specific for the grafted tissue by incubating the one or more serum samples with an antigen preparation made from cells or tissues of the donor obtained prior to transplantation;

(b) in parallel, incubating a pre-transplant serum sample from the recipient with the antigen preparation to determine a baseline level of antibodies specific for the grafted tissue;

(c) comparing the level of graft-specific antibodies in (a) and (b), wherein a significant increase in the level of antibodies in the one or more post-transplant serum samples is indicative of the immune response.

The grafted tissue is preferably a heart allograft.

The antigen preparation preferably comprises homogenized membrane fragments prepared from donor tissue, preferably heart or heart-associated tissue, most preferably tissue that comprises the posterior wall of the right atrium and/or a joining portion of vena cava.

In one embodiment, the homogenized membrane fragments are immobilized to a solid support.

The above testing preferably comprises an enzyme immunoassay or a light emitting immunoassay, most preferably, an ELISA.

Also provided is a method for early detection of an antibody response in a heart transplant recipient against the grafted heart tissue from a donor, comprising:

(a) testing by ELISA one or more serum samples from the recipient to measure the level of antibodies specific for the grafted heart tissue by incubating the one or more serum samples with an immobilized antigen preparation which comprises homogenized membrane fragments prepared from the donor heart or heart-associated tissue;

(b) in parallel, testing by ELISA a pre-transplant serum sample from the recipient by incubating the pre-transplant sample with the immobilized antigen preparation to determine a baseline level of antibodies specific for the grafted tissue; and

(c) comparing the level of graft-specific antibodies in (a) and (b), wherein a significant increase in the level of antibodies in the one or more post-transplant serum samples is indicative of the antibody response.

In this method, the donor heart or heart-associated tissue preferably comprises the posterior wall of the right atrium, and/or a joining portion of vena cava.

In one embodiment, the grafted tissue is a heart, kidney, liver, lung or pancreas allograft and the antigen preparation comprises Epstein Barr virus transformed donor lymphocytes immobilized in wells of the ELISA plate.

### DESCRIPTION OF THE PREFERRED EMBODIMENTS

In the following description, reference will be made to various methodologies known to those of skill in the art of immunology. Publications and other materials setting forth such known methodologies to which reference is made are incorporated herein by reference in their entireties as though set forth in full. Standard reference works setting forth the general principles of immunology include A. K. Abbas etal., Cellular and Molecular Immunology (Fourth Ed.), W.B. Saunders Co., Philadelphia, 2000; C. A. Janeway et al., Immunobiology. The Immune System in Health and Disease, Fourth ed., Garland Publishing Co., New York, 1999; Roitt, I. et al., Immunology, (current ed.) C.V. Mosby Co., St. Louis, Mo. (1999); Klein, J., Immunology, Blackwell Scientific Publications, Inc., Cambridge, Mass., (1990); Harlow, E. et al., Antibodies: A Laboratory Manual, Cold Spring Harbor Laboratory Press, Cold Spring Harbor, N.Y., 1988 or later edition).

Immunoassay methods are also described in Coligan, J. E. et al., eds., Current Protocols in Immunology, Sec. 2.4.1, Wiley-Interscience, New York, 1992(or current edition); Butt, W. R. (ed.) Practical Immunoassay: The State of the Art, Dekker, New York, 1984; Bizollon, Ch. A., ed., Monoclonal Antibodies and New Trends in Immunoassays, Elsevier, New York, 1984; Butler, J. E., ELISA (Chapter 29), In: van Oss, C. J. et al., (eds), IMMUNOCHEMISTRY, Marcel Dekker, Inc., New York, 1994, pp. 759-803; Butler, J. E. (ed.), Immunochemistry of Solid-Phase Immunoassay, CRC Press, Boca Raton, 1991.

The earliest event which may be clearly detectable in an immune response against an allograft is the production of antibodies against one or more antigens of the allograft. These antibodies can be detected by an enzyme immunoassay (EIA) such as an ELISA (Enzyme Linked Immunosorbent Assay) in which the allograft tissue serves as the source of immobilized antigen. Although previous studies measured antibody production against the allogeneic HLA antigens identified by tissue typing (Reed et al. Transplantation 61: 566-72, 1996), the use of donor tissue as test antigen is much more sensitive, since such an assay measures the full range antibody production to all alloantigens on the graft that are now present in immobilized form on the assay plate. Importantly, the alloantigens of the allograft do not have to be characterized for this purpose. The level of antibodies against the allograft antigens is determined relative to the baseline activity of antibodies in pre-transplantation serum samples to the same allograft preparation. The full range of anti-allograft antibodies in heart recipients is measured with the donor heart tissue as the solid phase antigen. In recipients of other, non-cardiac allografts, in whom the transplanted tissue may not be available for such analysis, the solid phase antigen may be obtained in other forms, e.g., as a lymphoid cell line generated from the graft donor's blood lymphocytes.

In another embodiment, the immune response to an the allograft is can be also evaluated by analysis of T cell reactivity to the full range of alloantigens expressed on the heart tissue membranes in heart recipients (or on a lymphoid cell line generated from the allograft donor, in the case where donor tissue (e.g., kidney) is not available.

Monitoring Antibody Responses to Heart Transplants—

This invention exploits the fact that not all of the donor's heart is used for grafting. The posterior wall of the donor's right atrium and the joining portion of the vena cava and occasionally other parts of the graft are removed prior to the joining of the heart to the posterior wall of the recipient's right atrium. Instead of discarding these portions of the donor's heart or associated tissue, they will be sent to the laboratory performing the immunological tests. In the laboratory, the specimens are minced and homogenized in a tissue homogenizer, into fragments with size ranging, but not limited to 1-100 μm. The resulting suspension is washed by centrifugation and re-suspended in saline. This membrane fragment suspension is frozen in aliquots at a preferred concentration of about 10 mg/ml. This suspension serves as the source of antigens that are tested in vitro for immune reactivity against the heart graft.

In one embodiment, this membrane suspension is dried overnight in wells of a plastic microplate (termed ELISA wells) at a preferred concentration of about 2 mg/ml in saline. The concentration of the heart fragmented membranes is not limited to 2 mg/ml and the tissue material may be suspended in other buffers, such as phosphate buffer saline (PBS) or carbonate buffer, as well. The drying of the membranes to the ELISA wells results in very strong adhesion of the membranes to the wells making them particularly useful as a solid phase antigen. Adhesion of the donor heart fragmented membrane suspension to ELISA wells also may be accomplished by a variety of other methods in which antigenic material is immobilized for use in EIA's. For a description of the use of other homogenized human tissues preparations as solid phase antigens in ELISA see, for example, Galili and LaTemple, Immunol.Today 18:281-285, 1997) and Galili et al., Gynecol Oncol. 90:100-108, 2003) which describes antibody binding to tumor membranes. The ELISA plates are blocked using conventional methods, e.g., using 1% bovine serum albumin in PBS for a period, of about 2 h or longer. Blocking can be achieved using other blocking solutions including, but no limited to powdered milk.

After completion of the blocking, serum samples are dispensed into these wells at serial two fold dilutions. A pre-transplantation serum sample is used in parallel for comparing the antibody activity at each time point with the pre-transplantation baseline. After an incubation of about 2 h, the ELISA plates are washed to remove the serum and any unbound antibodies. Binding of antibodies of the IgM, IgG or IgA isotypes is determined by the use of peroxidase conjugated isotype-specific anti-human Ig antibodies (anti human IgM, anti-human IgG, anti-human IgA), or anti-human total Ig secondary antibodies. The secondary anti-human Ig antibodies may be conjugated to other enzymes that catalyze chromogenic reactions, or coupled to light- or irradiation-emitting substances (referred to also as a “light emitting immunoassay”). If an antibody response against the graft is developing, anti-graft antibodies will be detected in the serum over time in comparison with the pre-transplant baseline activity. An increase of≧4 fold in IgM, IgG or IgA antibodies that bind to the donor tissue material in the ELISA wells is considered significant.

Monitoring Antibody Responses to Kidney Transplant

The analysis of antibody response to the transplanted kidney is performed by the same method as that described above using kidney tissue homogenate as solid phase antigen. However, if no donor kidney tissue is available for the assay, any other donor solid tissue will be suitable. Such a tissue specimen is homogenized and prepared as described above. Because of the high likelihood that no other donor tissue from living donors may be available for use as immobilized antigen, long term cell lines from the donor, such as B cells transformed by Epstein Barr virus (EBV) may be used as the source of the solid phase antigenic material. Methods for preparation of such B cell lines is routine in the art. See, for example, Galili et al., Blood 102:229, 2003). EBV is obtained, for example, from cultures of the marmoset B cell line B95.8, which constitutively secrete EBV virions into the medium. Transformed B cells are expanded and frozen in solutions containing dimethyl sulfoxide (DMSO) to maintain viability.

Transformed lymphocytes are washed and allowed to dry in ELISA wells at any of a range of concentrations, preferably about 2×10⁶ cells/ml in 50 μl volumes of PBS or other suitable buffer. These transformed donor lymphocytes serve as solid phase antigen in ELISA or in any other immunoassay measuring anti-donor antibodies present in the recipient's serum. As with the cardiac donor tissue, an increase of≧4 fold in IgM, IgG, IgA (or total Ig) antibodies that bind to the to the donor's transformed lymphocytes in the ELISA wells, in comparison to the pre-transplant serum is considered significant.

Monitoring Cellular (T Cell) Immune Response to Heart Transplants

Monitoring the cellular immune response to the cardiac allograft is done by co-incubation of the recipient's peripheral blood mononuclear cells (PBMC) with antigen preparations (preferably suspensions of fragmented donor heart tissue) followed by measurement of T lymphocyte activation as a result of this exposure. T cell activation can be measured as T cell proliferation and/or cytokine production. These methods are routine in the art, and are described only briefly below. In all cases, pre-transplant PBMC are obtained from the patient and cryopreserved using conventional techniques. These cells serve as controls for all post-transplant studies and are used after appropriate thawing under conditions that are well-known in the art to preserve function.

Cell proliferation-PBMC are isolated from the recipient's blood in the presence of anti-coagulant. The PBMC are incubated at a preferred concentration of about 10⁶ cells/ml with the donor heart fragmented membranes at a preferred concentration of about 1 mg/ml for about 5 days at 37° C. Positive control PBMC are incubated with an optimal concentration of a T cell mitogen such as concanavalin A (e.g.,, 1 μg/ml) and negative controls are incubated in medium with no membrane preparation or a control membrane preparation from an unrelated source or from the recipient. After incubation for am adequate period, e.g., 4 days, a radionuclide such as ³H-thymidine is added at a preferred concentration of about 1 μCi/ml (Tanemura et al., J Clin Invest 105: 301, 2000). The cells are harvested after 24 h and ³H-thymidine uptake is determined. There is a direct correlation between the stimulation of T lymphocytes reacting to the donor heart alloantigens and the uptake of ³H-thymidine. It is expected that this uptake will increase in the course of an anti-heart immune response by at least two-fold in comparison with the stimulation observed with lymphocytes obtained prior to transplantation. Proliferation of lymphocytes may be also determined by flow cytometry in which cells are permeabilized and stained with propidium iodide. Proliferating cells incorporate up to two fold higher amounts of propidium iodide because of doubling in the amount of DNA prior to cell division (Venkateswaran et al, J Urol. 2002; 168: 1578-1582). The proportion of dividing cells is measured as an increase in propidium iodide staining, determined by fluorescence intensity.

To measure cytokine production, the PBMC are incubated for a preferred interval of about 48 h at 37° C. at a preferred concentration of about 5×10⁶ cells/ml in U-shaped wells with a sufficient concentration, e.g., 0.1 or 1 mg/ml donor membrane preparation in medium containing recombinant IL2 at a preferred concentration of about 100 units/ml. Subsequently, the cells are plated in ELISPOT wells coated with anti-IFNγ or anti-IL4. The cells are plated in these wells at preferred concentrations of between about 0.3 and about 5×10⁶ cells/ml, in presence of preferred concentration of about 1.0 mg/ml of the heart fragmented membranes and with IL2 as above. After overnight incubation the ELISPOT wells are washed to remove PBMC and fragmented membranes. Spots indicating individual TH1 cells (secreting interferon-γ (IFNγ)), or TH2 cells (secreting IL4) are developed with peroxidase coupled antibodies specific for IFNγ or IL4, respectively (Gebauer et al., Am J Transplant. 2002; 2: 857-866). Negative control wells contain PBMC but no stimulatory fragmented membranes and generate a background level of spots. Positive control wells contain PBMC and Concanavalin-A as above. This lectin is a polyclonal T cells activator. A developing cellular immune response against the graft is accompanied by increases in the numbers of antigen-specific TH1 and/or TH2 cells. Overnight co-incubation of PBMC with donor membranes will result in the stimulation of these cells, which can be enumerated as the number of spots of IFNγ or IL4. representing TH1 or TH2 cells, respectively. An alternative way to detect these stimulated lymphocytes is by staining for intracellular cytokines which requires permeabilization of the mononuclear cells and staining using with labeled monoclonal antibodies to IFNγ or to IL4 (Koehne G. et al. Blood 99:1730, 2002).

Monitoring cellular (T cell) immune response to kidney transplants—The assays are performed as described above using fragmented kidney or other donor tissue or a donor cell line such as EBV-transformed B lymphocytes as antigen.

The tests for antibody and T cell response in recipients of allografts other than heart or kidney may be performed by the methods described herein. The cell membranes used as a source of antigen may be from donor tissue homogenates obtained or from donor lymphoid cell lines generated by EBV transformation of the donor's B lymphocytes.

### List of References Cited Above

DeBruyne L A, Ensley R D, Olsen S L, Taylor D O, Carpenter B M, Holland C, Swanson S, Jones K W, Karwande S V, Renlund D G, et al. Increased frequency of alloantigen-reactive helper T lymphocytes is associated with human cardiac allograft rejection.: Transplantation 1993; 56: 722-727.

Galili U, Anaraki F, Thall A, Hill-Black C, Radic M. One percent of circulating B lymphocytes are capable of producing the natural anti-Gal antibody. Blood 1993; 82: 2485-2493.

Galili U, Chen Z, DeGeest K. Expression of α-gal epitopes on ovarian carcinoma membranes to be used as a novel autologous tumor vaccine. Gynecologic Oncology 2003; 90: 100-108.

Galili U, LaTemple D C. The natural anti-Gal antibody as a universal augmenter of autologous vaccine immunogenicity. Immunology Today, 18:281-285, 1997.

Gebauer B S, Hricik D E, Atallah A, Bryan K, Riley J, Tary-Lehmann M, Greenspan N S, Dejelo C, Boehm B O, Hering B J, Heeger P S. Evolution of the enzyme-linked immunosorbent spot assay for post-transplant alloreactivity as a potentially useful immune monitoring tool. Am J Transplant. 2002; 2: 857-866.

Grebe S O, Mueller T F. Immune monitoring in organ transplantation using neopterin. Curr Drug Metabol 2002; 3: 189-202.

Hariharan S, Johnson C P, Bresnahan B A, Taranto S E, McIntosh M J, Stablein D. Improved graft survival after renal transplantation in the United States, 1988 to 1996. N Engl J Med 2000; 342: 605-612.

Hetzer R, Potapov E V, Muller J, Loebe M, Hummel M, Weng Y, Warnecke H, Lange P E. Daily noninvasive rejection monitoring improves long-term survival in pediatric heart transplantation. Ann Thorac Surg. 1998; 66: 1343-1349.

Kemkes B M, Schutz A, Engelhardt M, Brandl U, Breuer M. Noninvasive methods of rejection diagnosis after heart transplantation. J Heart Lung Transplant. 1992; 11: S221-231.

Khoss A E, Balzar E, Steger H, Howanietz H, Wladika W, Hamilton G, Woloszczuk W. Child Nephrol Urol 1988; 9: 4649.

Kimball P M, Radovancevic B, Isom T, Spickard A, Frazier O H. The paradox of cytokine monitoring-predictor of immunologic activity as well as immunologic silence following cardiac transplantation. Transplantation 1996; 61: 909-915.

Koehne G, Smith K M, Ferguson T L, Williams R Y, Heller G, Pamer E G, Dupont B, O'Reilly R J. Quantitation, selection, and functional characterization of Epstein-Barr virus-specific and alloreactive T cells detected by intracellular interferon-gamma production and growth of cytotoxic precursors. Blood 2002; 99: 1730-1740.

Li B, Hartono C, Ding R, Sharma V K, Ramaswamy R, Qian B, Serur D, Mouradian J, Schwartz J E, Suthanthiran M. Noninvasive diagnosis of renal-allograft rejection by measurement of messenger RNA for perforin and granzyme B in urine. N Engl J Med 2001; 344: 947-954.

Loonen L, Vaessen L, Balk A, Groeneveld K, Mochtar B, Jutte N, Claas F, Weimar W. Long-term survival of heart grafts in the presence of donor-specific cytotoxic T-cell precursors (CTLp) in the peripheral blood. Transplant Int 1994; 7: 596-598.

Reader J A, Burke M M, Counihan P, Kirby J A, Adams S, Davies M J, Pepper J R. Noninvasive monitoring of human cardiac allograft rejection. Transplantation 1990; 50: 29-33.

Reed E F, Hong B, Ho E, Harris P E, Weinberger J, Suciu-Foca N. Monitoring of soluble HLA alloantigens and anti-HLA antibodies identifies heart allograft recipients at risk of transplant-associated coronary artery disease. Transplantation 1996; 61: 566-572.

Salaman J R. Monitoring of rejection in renal transplantation. Immunol Lett 1991; 29: 139-142.

Tanemura M, Yin D, Chong A S, Galili U. Differential immune responses to alpha-gal epitopes on xenografts and allografts: implications for accommodation in xenotransplantation. J Clin Invest 2000; 105: 301-310.

Venkateswaran V, Fleshner N E, Klotz L H. Modulation of cell proliferation and cell cycle regulators by vitanin E in human prostate carcinoma cell lines. J Urol. 2002; 168: 1578-1582.

All the references cited above are incorporated herein by reference in their entirety, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

What is claimed is:

1. A method for early detection of an immune response in a transplant recipient against the grafted tissue or organ from a donor, comprising:
(a) testing one or more serum samples from said recipient obtained early after transplantation to measure the level of antibodies specific for said grafted tissue by incubating said one or more serum samples with an antigen preparation made from cells or tissues of the donor obtained prior to transplantation;
(b) in parallel, incubating a pre-transplant serum sample from said recipient with said antigen preparation to determine a baseline level of antibodies specific for said grafted tissue;
(c) comparing the level of graft-specific antibodies in (a) and (b),
wherein a significant increase in the level of antibodies in said one or more post-transplant serum samples is indicative of said immune response.

2. The method of claim 1 wherein said grafted tissue is a heart allograft.

3. The method of claim 2 wherein said antigen preparation comprises homogenized membrane fragments prepared from donor tissue.

4. The method of claim 3 wherein the donor tissue comprises the posterior wall of the right atrium and/or a joining portion of vena cava.

5. The method of claim 3 wherein said homogenized membrane fragments are immobilized to a solid support.

6. The method of claim 1 wherein said testing comprises an enzyme immunoassay or a light emitting immunoassay.

7. The method of claim 6 wherein said enzyme immunoassay is an ELISA.

8. A method for early detection of an antibody response in a heart transplant recipient against the grafted heart tissue from a donor, comprising:
(a) testing by ELISA one or more serum samples from said recipient to measure the level of antibodies specific for said grafted heart tissue by incubating said one or more serum samples with an immobilized antigen preparation which comprises homogenized membrane fragments prepared from the donor heart or heart-associated tissue;
(b) in parallel, testing by ELISA a pre-transplant serum sample from said recipient by incubating the pre-transplant sample with said immobilized antigen preparation to determine a baseline level of antibodies specific for said grafted tissue; and
(c) comparing the level of graft-specific antibodies in (a) and (b), wherein a significant increase in the level of antibodies in said one or more post-transplant serum samples is indicative of said antibody response.

9. The method of claim 8, wherein the donor heart or heart-associated tissue comprises the posterior wall of the right atrium, and/or a joining portion of vena cava.

10. The method of claim 8 wherein said grafted tissue is a heart, kidney, liver, lung or pancreas allograft and the antigen preparation comprises Epstein Barr virus-transformed donor lymphocytes immobilized in assay plate wells.
